(19) United States
(12) Patent Application Publication
(10) Pub. No.: US 2005/0096611 A1
(43) Pub. Date: May 5, 2005

(54) Multi-adhesive medical appliance

(76) Inventors: Brian C. Stoyer, Langhorne, PA (US); Paul Armour, Belle Mead, NJ (US)

Correspondence Address: BRISTOL-MYERS SQUIBB COMPANY, 100 HEADQUARTERS PARK DRIVE, SKILLMAN, NJ, 08558, US

(21) Appl. No.: 10/970,790

(22) Filed: Oct. 21, 2004

Related U.S. Application Data
(60) Provisional application No. 60/515,474, filed on Oct. 30, 2003.

(51) Int. Cl. A61F 5/44

(52) U.S. Cl. 604/332

(57) ABSTRACT

A medical appliance designed to adhere to the skin proximate a stoma or other open wound with an adhesive layer including two or more different composition pressure sensitive adhesives. One adhesive is skin friendly, having excellent tack properties, and painless removability. The other adhesive is flexible and comfortable to wear, has high moisture tolerance and the ability to swell to create a seal around the stoma. It has minimal cold flow and will not discolor significantly when sterilized.

This application claims the benefit of U.S. Provisional Application No. 60/515,474, filed 30 Oct., 2003 and is hereby incorporated by reference in its entirety.

### FIELD OF THE INVENTION

The present invention relates to an adhesive medical appliance designed to be removably mounted on the skin as part of an ostomy device or wound care product and more particularly to such an appliance which has the attributes of two or more types of pressure sensitive adhesives, one of which has excellent initial wet tack characteristics and can be painlessly removed from the skin, and the other of which is flexible and comfortable to wear, exhibits minimal cold flow, can withstand high moisture conditions without disintegrating, has the ability to swell to create a seal around the stoma in a controlled fashion and can be sterilized without significant color change.

### BACKGROUND OF THE INVENTION

Ostomy devices and wound care products such as bandages must adhere to and be removable from the skin proximate the stoma or other open wound. Accordingly, adhesives with special attributes that make them suitable for use on the skin proximate the stoma or other open wound have been developed for such medical appliances.

Certain of these adhesives are skin friendly, having the capability of transferring moisture such as perspiration and wound exudate away from the skin. They have excellent initial wet tack qualities and permit non-painful removal of the appliance, even after short wear times. They are formed mainly of natural ingredients and have cold flow characteristics that permit the adhesive to “fill in” any skin irregularities, providing better adhesion to the skin.

One such skin friendly adhesive material that has been found to work particularly well is a pressure sensitive polyisobutylene (PIB) adhesive having intimately dispersed therein a water soluble or swellable hydrocolloid or mixture of hydrocolloids. That material is disclosed in U.S. Pat. No. 3,972,328, issued Aug. 3, 1976 to James Chen, and owned by the Bristol-Myers Squibb Company, Lawrenceville, N.J. It is commercially used on many medical products sold by the ConvaTec Division of Bristol-Myers Squibb Company.

However, the cold flow properties of the adhesive disclosed in the aforementioned Chen patent may permit flow to the point of creating undermining liquid flow channels, sometimes causing the adhesive to disintegrate when coming in contact with liquid from the stoma or wound. Thicker versions of appliances with that adhesive tend to be somewhat rigid and hence may be less comfortable to wear. That adhesive may be susceptible to absorbing liquid, drying out and subsequently creating hard crystallized edges over time.

Further, the adhesive disclosed in the Chen patent does not have the ability to swell creating a seal around the stoma. It may also exhibit some negative appearance characteristics after sterilization. After gamma irradiation, it has a propensity to excessively cold flow, discolor and, with time, become rigid and brittle.

For those reasons, other adhesive materials have been developed for use in medical appliances of this type. Those adhesives tend to be more flexible and thus more comfortable to wear. They are more moisture tolerant and have the ability to swell to create a seal around the stoma in a controlled fashion, minimizing effluent contact with the persistomal skin. Those adhesives also exhibit less cold flow and minimal product erosion when exposed to effluent. Further, the adhesives exhibit little color or cold flow after gamma sterilization.

Such adhesive materials are disclosed in U.S. Pat. No. 4,551,490 issued Nov. 5, 1985 to Doyle et al., U.S. Pat. No. 4,538,603, issued Sep. 3, 1985 to Pawelchak et al., U.S. Pat. No. 4,728,642, issued Mar. 1, 1988 to Pawelchak et al. and U.S. Pat. No. 5,006,401, issued Apr. 9, 1991 to Frank, all of which are also owned by the Bristol-Myers Squibb Company of Lawrenceville, N.J. Those adhesives are also commercially used in medical products sold by the ConvaTec Division of Bristol-Myers Squibb Company.

Those patents disclose medical grade pressure sensitive compositions including a homogeneous mixture of one or more polyisobutylenes or blends of one or more polyisobutylenes and butyl rubber, one or more styrene radial or block type copolymers, mineral oil, one or more water soluble hydrocolloid gums, and a tackifier. They may also include one or more water swellable cohesive strengthening agents, an anti-oxidant and various other optional ingredients.

Those rubber based hydrocolloid adhesives contain a tackifier, unlike the adhesive material disclosed in the Chen patent. The tackifier is required in the formulation to create the necessary adhesion to the skin. The use of a tackifier can substantially increase adhesion after prolonged skin contact. Because of this adhesive ability, appliances with those adhesives typically need not be removed from the skin for at least three days after attachment to the skin. Such an aggressive nature and the use of a tackifier that does not necessarily support excellent “wet” tack, make those adhesives better suited as a secondary layer, located away from the skin.

Thus, each of the above types pressure sensitive hydrocolloid adhesives have certain qualities that make them excellent for use in medical appliances designed to adhere to the skin and each has drawbacks that make it less desirable for such application. The hydrocolloid adhesive disclosed in the Chen patent is skin friendly, has great initial dry and wet tack, and can be removed from the skin painlessly, and thus is best used where skin contact is required. The hydrocolloid adhesives disclosed in Doyle et al., Pawelchak et al. and Frank patents noted above have reduced wet tack, are more aggressive in short wear periods and are best used to give the appliance enhanced flexibility and comfort, increased moisture tolerance, the ability to swell to create a seal around the stoma in a controlled fashion and can be sterilized without discoloration.

It is, therefore, a prime object of the present invention to provide a multi-layered adhesive medical appliance that has the attributes of a wet tack pressure sensitive adhesive for use adjacent the skin, and of a flexible, comfortable, moisture tolerant adhesive that resists degradation after sterilization, and is capable of creating a seal around the stoma in a controlled fashion, for use away from the skin.

It is another object of the present invention to provide a multi-layered adhesive medical appliance in which different adhesives are utilized such that the advantageous qualities of each type of adhesive are maximized and the disadvantages of each type of adhesive are minimized in order to provide an improved ostomy device or wound care product.

### SUMMARY OF THE INVENTION

In accordance with one aspect of the present invention, a medical appliance adapted to adhere to the skin is provided. The appliance includes a film layer, a first hydrocolloid adhesive and a second hydrocolloid adhesive. The composition of the second hydrocolloid adhesive is different than the composition of the first hydrocolloid adhesive.

In one preferred embodiment, the second hydrocolloid adhesive is situated between the film layer and the first hydrocolloid adhesive. In a second preferred embodiment, the film layer is situated between the first hydrocolloid adhesive and the second hydrocolloid adhesive.

The appliance has a first surface adapted to adhere to the skin. In both preferred embodiments, the first hydrocolloid adhesive forms the first appliance surface.

The appliance has a second surface. In the first preferred embodiment, the film layer forms the second surface. In the second preferred embodiment, the second hydrocolloid adhesive forms the second appliance surface.

A removable protective layer is provided adjacent the first appliance surface. The removable protective layer preferably comprises a silicone release coated substrate.

The first hydrocolloid adhesive forms a first adhesive layer. The second hydrocolloid adhesive forms a second adhesive layer. Preferably, the second adhesive layer is thicker than the first adhesive layer.

The film layer preferably comprises polyethylene or other flexible film. The film layer may have a smooth surface or the surface of the film layer may be embossed.

The first hydrocolloid adhesive preferably includes a polyisobutylene having dispersed therein a water soluble or swellable hydrocolloid or mixture of hydrocolloids.

The second hydrocolloid adhesive preferably includes a homogeneous mixture of one or more polyisobutylenes or blends of one or more polyisobutylenes and butyl rubber, one or more styrene radial or block type copolymers, mineral oil, one or more water soluble hydrocolloid gums, and a tackifier.

The second hydrocolloid adhesive layer preferably also includes one or more swellable cohesive strengthening agents and an anti-oxidant.

### BRIEF DESCRIPTION OF THE DRAWINGS

To these and such other objects that may hereinafter appear, the present invention relates to a multi-layer adhesive medical device as described in the following specification and recited in the annexed claims, taken together with the accompanying drawing, wherein like numerals refer to like parts and in which:

FIG. 1 is an isometric view of the adhesive wafer portion of a conventional ostomy device;

FIG. 2 is a cross-sectional view of the product of FIG. 1;

FIG. 3 is a cross-sectional view of the first preferred embodiment of the medical device of the present invention;

FIG. 4 is a cross-sectional view of a second preferred embodiment of the medical appliance of the present invention; and

FIG. 5 is a cross-sectional view of a third preferred embodiment of the medical appliance of the present invention.

### DETAILED DESCRIPTION OF THE DRAWINGS

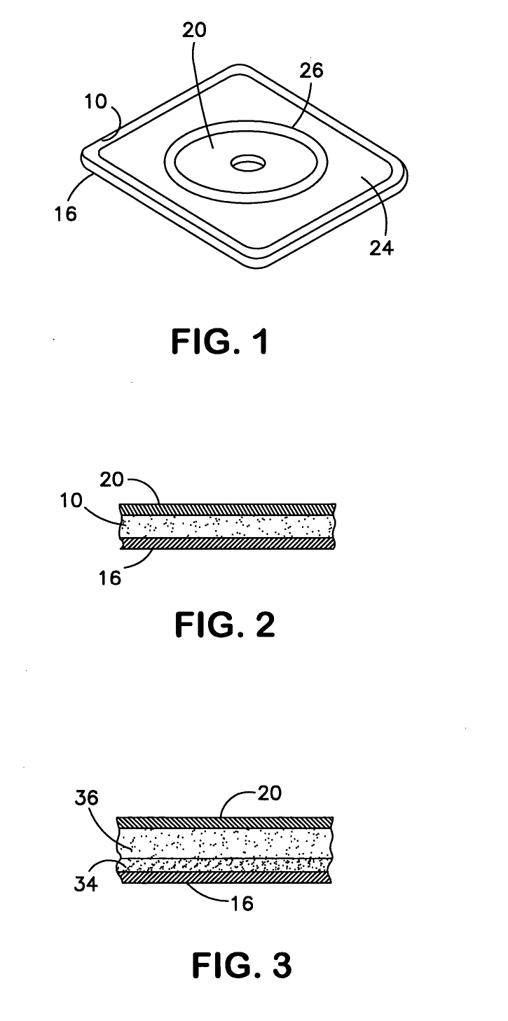

FIG. 1 illustrates a conventional adhesive wafer portion of an ostomy device. As seen in the cross-sectional view of FIG. 2, this wafer consists of a pressure sensitive adhesive layer 10 formed of a single adhesive material. That adhesive material may be, for example, the material disclosed in either the aforementioned Chen patent or that disclosed in any of the aforementioned Doyle et al., Pawelchak et al. or Frank patents. Silicone release paper 16 is situated adjacent the skin facing (bottom) surface of the adhesive layer 10. Polyethylene film 20 is situated on the other (top) surface of the adhesive layer 10.

In a separate process not illustrated and as is well known in the art, see, for example, U.S. Pat. Nos. 4,460,363, 4,465,486, 4,419,174, 4,701,169, 4,775,374, 5,951,533, 6,602,232, incorporated herein by reference, the single adhesive layer wafer shown in FIG. 1 is attached to a waste collection receptacle such as pouch or bag, either permanently by heat or sonic welding or similar means, or by utilizing inter-engaging coupling rings that permit the collection receptacle to be detached from the wafer. This allows the collection receptacle to be emptied and cleaned, without removing the wafer from the skin, and thereafter to be reattached.

The adhesive wafer is attached to the skin surrounding the stoma after the central opening of the wafer is enlarged to fit around the stoma and release paper 16 is removed from the surface of the adhesive layer. A transparent protective plastic shield 24 may be affixed to film 20 by a ring weld 26, if desired.

The resulting appliance is illustrated in cross-section in FIG. 3. As seen in that figure, the skin friendly first hydrocolloid adhesive layer 34 is preferably substantially thinner than the second hydrocolloid adhesive layer 36.

The resulting product will appear essentially as illustrated in FIG. 3 if the adhesives are extruded into distinct layers 34 and 36. However, in some instances, it may be preferable to mix the first hydrocolloid adhesive and the second hydrocolloid adhesive before they are extruded. In that case, the resulting product will appear similar to the wafer illustrated in FIG. 2, except that adhesive layer 10 will be made up of a mixture of the two different composition hydrocolloid adhesives.

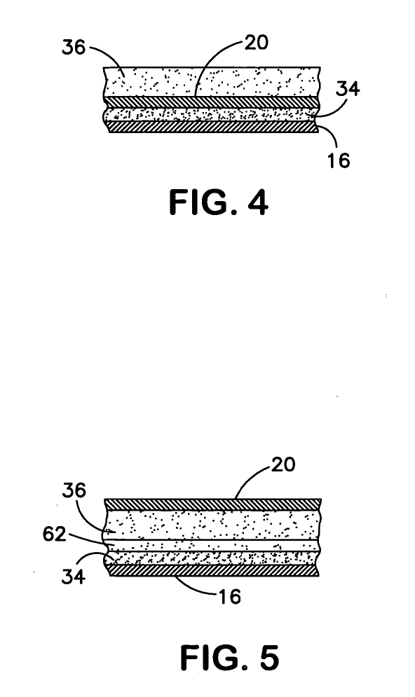

FIG. 4 illustrates the cross-sectional structure of the appliance that includes silicone release film 16, a first hydrocolloid adhesive layer 34, a second hydrocolloid adhesive layer 36 and a film layer 20 situated between the two adhesive layers 34 and 36. A second film layer (not shown) may be situated on top of the second hydrocolloid adhesive layer 36.

FIG. 5 illustrates the cross-sectional embodiment of an appliance having an adhesive layer 62 is situated between the first and second hydrocolloid adhesive layers 34,36. Layer 62 is formed from the mixture of the first hydrocolloid adhesive and the second hydrocolloid adhesive. The relative thicknesses of the adhesive layers 34, 36 and 62 can be adjusted, as required. A film layer 20 is on top of the second hydrocolloid adhesive layer 36.

It is possible that when the second hydrocolloid adhesive layer 36 of the multi-adhesive ostomy embodiments described herein comprises the majority of the adhesive structure at least in the area around the stoma, the adhesive near the stoma is capable of being molded around the stoma so as to form a seal.

Alternatively, it is possible to combine the multi-adhesive hydrocolloid structure described herein with a moldable adhesive such as the type described in U.S. application Ser. No. 10/188,535, incorporated herein by reference. The moldable adhesive would be present around the stomal opening and a multi-adhesive portion would surround the moldable adhesive.

It should now be appreciated that the present invention relates to a medical appliance designed to adhere to the skin proximate a stoma or other open wound that has an adhesive layer including two or more different composition pressure sensitive adhesives, each having distinct qualities that make it desirable for use in this type of product. One adhesive is skin friendly, having excellent tack properties and can be removed from the skin painlessly, even after short wear times. The other adhesive is flexible and comfortable to wear. It has high moisture tolerance and the ability to swell to create a seal around the stoma. It has minimal cold flow and will not discolor significantly when sterilized.

1. A medical appliance adapted to adhere to the skin comprising a film layer, a first hydrocolloid adhesive and a second hydrocolloid adhesive, said second hydrocolloid adhesive having a different composition than said first hydrocolloid adhesive.

2. The medical appliance of claim 1 wherein said second hydrocolloid adhesive is situated between said film layer and said first hydrocolloid adhesive.

3. The medical appliance of claim 1 wherein said appliance has a first surface adapted to adhere to the skin and wherein said first hydrocolloid adhesive comprises said first appliance surface.

4. The medical appliance of claim 1 wherein said appliance has a second surface and wherein said film layer comprises said second appliance surface.

5. The medical appliance of claim 1 further comprising a removable protective layer.

6. The medical appliance of claim 5 wherein said removable protective layer is situated adjacent said first hydrocolloid adhesive.

7. The medical appliances of claim 5 wherein said removable protective layer comprises silicone release material on a substrate.

8. The medical appliance of claim 1 wherein said first hydrocolloid adhesive forms a first adhesive layer and said second hydrocolloid adhesive forms a second adhesive layer.

9. The medical appliance of claim 8 wherein said second adhesive layer is thicker than said first adhesive layer.

10. The medical appliance of claim 1 wherein said film layer comprises polyethylene or other flexible material.

11. The medical appliance of claim 1 wherein said first hydrocolloid adhesive comprises a polyisbobutylene having dispersed therein a water soluble or swellable hydrocolloid or mixture of hydrocolloids.

12. The medical appliance of claim 1 wherein said second hydrocolloid adhesive comprises a homogeneous mixture of one or more polyisobutylenes or blends of one or more polyisobutylenes and butyl rubber, one or more styrene radial or block type copolymers, mineral oil, one or more water soluble hydrocolloid gums, and a tackifier.

13. The medical appliance of claim 12 wherein said second hydrocolloid adhesive layer further comprises one or more swellable cohesive strengthening agents and an anti-oxidant.

14. The medical appliance of claim 1 wherein said film layer can be embossed.

15. The medical appliance of claim 1 wherein said film layer is situated between said first hydrocolloid adhesive and said second hydrocolloid adhesive.

16. The medical appliance of claim 1 wherein said adhesive layer comprises a mixture of said first hydrocolloid adhesive and said second hydrocolloid adhesive.

17. The medical appliance of claim 1 wherein said first hydrocolloid adhesive comprises a polyisbobutylene having dispersed therein a water soluble or swellable hydrocolloid or mixture of hydrocolloids and wherein said second hydrocolloid adhesive comprises a homogeneous mixture of one or more polyisobutylenes or blends of one or more polyisobutylenes and butyl rubber, one or more styrene radial or block type copolymers, mineral oil, one or more water soluble hydrocolloid gums, and a tackifier.

18. The medical appliance of claim 17 wherein said first hydrocolloid adhesive and said second hydrocolloid adhesive are situated on the same side of said film layer.

19. The medical appliance of claim 17 wherein said first hydrocolloid adhesive and said second hydrocolloid adhesive are located on opposite sides of said film layer.

20. The medical appliance of claim 8 further comprising a third adhesive layer formed of a mixture of said first hydrocolloid adhesive and said second hydrocolloid adhesive.

21. The medical appliance of claim 20 wherein said third hydrocolloid adhesive layer is interposed between said first hydrocolloid adhesive layer and said second hydrocolloid adhesive layer.

22. The medical appliance of claim 1 further comprising a stomal opening wherein said second hydrocolloid adhesive comprises a majority of said hydrocolloid adhesives and said adhesives are moldable around said stomal opening.

23. The medical appliance of claim 1 further comprising a stomal opening, a moldable adhesive portion around said stomal opening, and an adhesive portion having a first and second hydrocolloid adhesive around said moldable adhesive portion.
